(19) United States
(12) Patent Application Publication
(10) Pub. No.: US 2015/0297676 A1
(43) Pub. Date: Oct. 22, 2015

(54) COMPOSITION COMPRISING GRIM-19 AS ACTIVE INGREDIENT FOR PREVENTING OR TREATING OBESITY OR LIPID-RELATED METABOLIC DISEASES

(71) Applicant: Catholic University Industry Academic Cooperation Foundation, Seoul (KR)

(72) Inventors: Mi-La CHO, Seoul (KR); Joo-Yeon Jhun, Seoul (KR); Seon-Yeong Lee, Gyeonggi-do (KR); Yeong-Mi Mun, Seoul (KR); Jun-Geol Ryu, Gangwon-do (KR); Jae-Kyeong Byun, Chungcheongbuk-do (KR); Hye-Jin Son, Seoul (KR); Eun-Kyung Kim, Seoul (KR); Eun-Ji Yang, Seoul (KR); Jeong-Hee Jeong, Seoul (KR)

(73) Assignee: Catholic University Industry Academic Cooperation Foundation, Seoul (KR)

(21) Appl. No.: 14/426,324

(22) PCT Filed: Sep. 2, 2013
(86) PCT No.: PCT/KR2013/007908

§ 371 Date: Mar. 5, 2015

(30) Foreign Application Priority Data
Sep. 5, 2012 (KR) 10-2012-0098140

(51) Int. Cl. A61K 38/17; G01N 33/68; C07K 14/47

(52) U.S. Cl. CPC A61K 38/1709; C07K 14/47; G01N 33/6872; G01N 2333/4704; G01N 2500/10; G01N 2500/04

(57) ABSTRACT

The present invention relates to a composition comprising GRIM-19 protein as an active ingredient for preventing or treating obesity or lipid-related metabolic diseases. More particularly, the present invention relates to a composition for preventing or treating obesity or lipid-related metabolic diseases wherein the composition comprises GRIM-19 protein as an active ingredient for preventing or treating metabolic diseases caused by immune response disorders or interactions between genetic, metabolic, or environmental complex factors. The GRIM-19 protein according to the present invention exhibits excellent effectiveness not only in reducing lipocytes and total cholesterol content in the body, but also in suppressing the differentiation of cytotoxic Th17 cells that produce and secrete inflammatory cytokines. Also, GRIM-19 has been found to have excellent effectiveness in regulating a STAT3 mediated inflammatory response in an inflammatory environment related to obesity. Thus, the present invention can be used for preparing a treatment agent and a functional food that can effectively treat obesity or lipid-related metabolic diseases.

### TECHNICAL FIELD

The present invention relates to a composition comprising Grim19 (gene associated with retinoid-IFN-induced mortality 19) protein as an active ingredient for the prevention or treatment of obesity or lipid-related metabolic diseases.

### BACKGROUND ART

In spite of the global effort to arouse attention to obesity and to understand the cause of obesity better to cope with it, obesity is still in fast increase (Mitchell et al., Implications for female fertility and obesity. Reproduction. 2005; 130:583-97). According to the report of Korea National Health and Nutrition Examination Survey in 2007, the number of domestic obese people increased 3% every year for the last 5 years and the population that are obese reached 32.7% (male: 33.1%, female: 32.2%) by the total population. Age-specifically, middle-aged classes that are obese is 44%, which is double the obese population of younger generation (22%). As obesity-related disease increases, socioeconomic costs reached approximately 1,823,900,000,000 Korean Won in 2007.

As the seriousness of obesity is realized, obesity itself is widely recognized as a disease. The judgment of the progress of obesity relies on the overweight or not. Precisely, if body fat is increased in a specific body part (for example, abdominal area), it can also be judged as obesity. Once diagnosed as obesity, the prevalence rate and the death rate of obese people are higher. Compared with normal body weight people, the death rate of diabetes among obese people is 4 times as high as that of the normal weight people and the death rate of liver cirrhosis is double. The death rate of cerebrovascular disease among obese people is 1.6 times higher and the death rate of coronary artery disease among obese people is 1.8 times higher.

According to the previous report, obesity is a reason of arteriosclerotic atheroma formation (Kunitomi M et al., Relationship between reduced serum IGF-I levels and accumulation of visceral fat in Japanese men. Int J Obes Relat Metab Disord. 2002; 26:361-9) and also a cause of many other diseases including hypertension, hyperlipidemia, immune disorder, and hyperinsulinemia. Resultingly, obesity increases the morbidity and mortality of cardiovascular disease (Hida K, et al., Visceral adipose tissue-derived serine protease inhibitor: a unique insulin-sensitizing adipocytokine in obesity. Proc Natl Acad Sci USA. 2005; 102:10610-5).

Obesity is a serious chronic syndrome characterized by excessive accumulation of fat, whose causes are known as many. The purposes of the treatment of obesity are two in general; one is to reduce weight by burning excessive fat and the second is to improve metabolic imbalance. The obesity patient, especially the abdominal obesity patient, is closely related to the pathological status such as X-syndrome (insulin resistance, type II diabetes, hypertension, and abnormal lipid metabolism), which can be a strong risk factor for early atherosclerosis, ischemic heart disease, and cerebrovascular disease.

Among many causes of obesity, the genetic factor takes more than 70% and the rest is filled with environmental factors including high fat diet and lack of exercise. Immune disorder is also a recent addition to the causes.

One of the cell groups playing an important role in immune system as a defense system against various pathogens is T-cell. T-cells are generated in thymus and then differentiated into the unique characteristic T-cells via a series of differentiation steps. The fully differentiated

T-cells are divided into two groups according to their functions: type I helper T-cells (Th1) and type II helper T-cells (Th2). Th1 is involved in cell mediated immunity, and Th2 is involved in humoral immunity. The immune system is balanced by the mutual check between the two groups not to be over-activated.

Most of immune-related diseases are therefore caused by the imbalance between the two immune cell groups. For example, when Th1 is abnormally over-activated, autoimmune disease can be developed. When Th2 is abnormally over-activated, immune disease caused by hypersensitivity reaction can be developed.

According to the recent research on the differentiation of Th1 cell, there is a newly identified cell group, which is the regulatory T cell (Treg) capable of regulating Th1 activity. So, studies to use the regulatory T cells (Treg) for the treatment of immune disease have been actively going on thereafter. Treg cells suppress the functions of abnormally activated immune cells to control inflammatory response. Therefore, the attempts to treat immune disease by increasing the activity of Treg cells have been reported.

Another cell group, in addition to the Treg cells, that is generated in the course of differentiation is Th17. Th17 cells are known to be generated in the course of differentiation of immature T cells, whose differentiation is similar to that of Treg cell. That is, the differentiations of both Treg cells and Th17 cells are accomplished in the presence of TGF-β. The difference between them is that Treg cells do not need IL-6 for the differentiation, while Th17 cells are only differentiated in the presence of both TGF-β and IL-6. The differentiated Th17 cells characteristically induce the secretion of IL-17.

Most common anti-obesity drugs are fat absorption inhibitors such as Xenical (Roche, Switzerland) and anorectic agents such as Meridia (Abbott, USA). These drugs however have such side effects as headache, raising blood pressure, and diarrhea, etc.

Therefore, it is required to develop a novel anti-obesity agent which is not expensive but is excellent in obesity treating effect without side effects.

### DISCLOSURE

### Technical Problem

The present inventors completed this invention by confirming that Grim19 has the effect of reducing body weight, reducing fat cells, lowering total cholesterol, glucose, and LDL-cholesterol, and converting white fat into brown fat, so as to prevent or treat effectively obesity or lipid related metabolic diseases.

It is an object of the present invention to provide a pharmaceutical composition for the prevention or treatment of obesity or lipid-related metabolic diseases comprising Grim19 protein as an active ingredient.

It is another object of the present invention to provide a pharmaceutical composition for the prevention or treatment of obesity of lipid-related metabolic diseases comprising the Grim19 protein of the invention or the polynucleotide encoding the same as an active ingredient.

It is also an object of the present invention to provide a method for screening a therapeutic agent for obesity or lipid-related metabolic diseases.

### Technical Solution

To achieve the above objects, the present invention provides a pharmaceutical composition for the prevention or treatment of obesity or lipid-related metabolic diseases comprising Grim19 protein as an active ingredient.

In a preferred embodiment of the present invention, the disease can be diabetes, hyperlipidemia, arteriosclerosis, hypertension, cardiovascular disease, fatty liver, obesity-mediated inflammatory disease, obesity-mediated autoimmune disease, or obesity-mediated cancer.

In a preferred embodiment of the present invention, the said Grim19 protein can be composed of the amino acid sequence represented by SEQ. ID. NO: 1.

In a preferred embodiment of the present invention, the said Grim19 demonstrates the effect of reducing body weight, reducing fat cells, lowering total cholesterol, and converting white fat into brown fat.

The present invention also provides a pharmaceutical composition for the prevention or treatment of obesity or lipid-related metabolic diseases comprising the Grim19 protein of the invention or the polynucleotide encoding the same as an active ingredient.

In a preferred embodiment of the present invention, the disease can be diabetes, hyperlipidemia, arteriosclerosis, hypertension, cardiovascular disease, fatty liver, obesity-mediated inflammatory disease, obesity-mediated autoimmune disease, or obesity-mediated cancer.

In a preferred embodiment of the present invention, the said polynucleotide can be composed of the nucleotide sequence represented by SEQ. ID. NO: 2 or NO: 3.

In a preferred embodiment of the present invention, the said polynucleotide is included in an expression vector.

In a preferred embodiment of the present invention, the said expression vector is a plasmid or a virus vector.

The present invention further provides a method for screening a therapeutic agent for obesity or lipid-related metabolic diseases comprising the steps of (a) culturing the Grim19 protein or the recombinant cells expressing the protein together with a candidate material; and (b) measuring the effect of the candidate material on the activity or the intracellular level of Grim19 protein.

In a preferred embodiment of the present invention, the activity or the intracellular level of Grim19 protein is measured by coimmunoprecipitation, RIA, ELISA, immunohistochemical method, Western blotting, or FACS.

In the present invention, it is firstly confirmed that Grim19 protein has the effect of preventing or treating obesity or lipid-related metabolic diseases. So, the present invention is characterized by providing a pharmaceutical composition for the prevention or treatment of obesity or lipid-related metabolic diseases comprising Grim19 protein as an active ingredient.

To develop a novel anti-obesity agent for treating metabolic disease, the present inventors paid attention to Grim19 gene. Grim19 was recently identified as the gene that related to cell death and interacted with STAT3 by yeast-2-hybrid screening (Zhang J, Yang J, Roy S K, Tininini S, Hu J, Bromberg J F. The cell death regulator Grim-19 is an inhibitor of signal transducer and activator of transcription 3. Proc Natl Acad Sci USA. 2003; 100:9342-9347). When the STAT3 related factors were first mentioned, PIAS3 and SOCS3 (suppressor of cytokine signaling 3) were confirmed as the STAT3 inhibiting feedback. Precisely, SOCS protein was confirmed to have the activity of inhibiting the ligand mediated response by suppressing JAKs and PIAS protein was known to have the activity of inhibiting STAT3 phosphorylation (Starr R, Hilton D J. Negative regulation of the JAK/STAT pathway. Bioessays 1999; 21:47-52).

Grim19 is also known to inhibit the STAT3 mediated transcription. Interestingly, Grim19 has been confirmed not to inhibit the phosphorylations of tyrosin and serine residues nor DNA binding thereof, unlike SOCS3 or PIAS3 (Chung C D, Liao J, Liu B, Rao X, Jay P, Berta P. Specific inhibition of Stat3 signal transduction by PIAS3. Science. 1997; 278:1803-1805). However, there was no report on the involvement of Grim19 in obesity or lipid-related metabolic diseases or on the mechanism involved in the treatment effect on them, either.

The present invention first confirmed that Grim19 protein could be used for the prevention or treatment of obesity or lipid-related metabolic diseases.

The above confirmation was re-assured in a preferred embodiment of the present invention. That is, when the Grim19 transgenic mouse (referred as “TG” hereinafter, the mouse transformed to over-express Grim19) was fed with high fat diet, the increase of body weight was significantly suppressed, compared with the normal mouse that was not transformed with Grim19 but induced with obesity with high fat diet, whose weight increase was almost as equal as that of the normal mouse fed with normal diet (see FIG. 1).

In a preferred embodiment of the present invention, Grim19 protein significantly reduced the number and the size of fat cells and the conversion of white fat into brown fat was also observed.

Particularly, white fat is responsible for changing excessive energy into triglyceride and deposit thereof. Therefore, when energy is over-generated by lack of exercise or overeating, the number of white fat cells increases, resulting in the obesity and changing the physical constitution to be apt to gain weight easily. In the meantime, brown fat plays a role in consuming the deposited energy by generating heat with that, so it is not responsible for the weight gaining and obesity.

Based on the facts above, since Grim19 reduces white fat inducing obesity but increases brown fat suppressing weight gaining, it can be effectively used for the prevention and treatment of obesity.

Grim19 also has the effect of reducing blood glucose, triglyceride, total cholesterol, AST, and ALT. AST and ALT have been used as the liver function test index. In obesity or lipid-related metabolic disease patients, AST and ALT are increased.

Therefore, the Grim19 of the present invention is not only effective in preventing and treating obesity but also in preventing and treating lipid-related metabolic diseases.

To disclose the correlation between obesity and inflammatory response, the present inventors performed FACS and spleen confocal histostaining to observe inflammatory cytokines in spleen cells of the normal and Grim19 TG mouse both fed with high fat diet. As a result, the Grim19 protein of the present invention was confirmed to regulate STAT3 mediated inflammatory response (see FIGS. 7a-7g).

The above result suggests that the Grim19 protein of the present invention has the effect of treating metabolic disease, and also preventing or treating obesity caused by immune dysregulation.

Therefore, the present invention can provide a pharmaceutical composition comprising Grim19 protein as an active ingredient for the prevention or treatment of obesity or lipid-related metabolic diseases. The Grim19 protein included in the pharmaceutical composition of the invention can contain any protein that has the practically same physiological activity to the protein. The protein having the practically same physiological activity to Grim19 protein includes functional equivalents and functional derivatives.

The “functional equivalent” herein indicates the transformed amino acid sequence with substitution, defect or addition of a part of or the whole natural amino acid sequence, which still has the same physiological activity to Grim19 protein. The “functional derivative” herein indicates the modified protein designed to increase or decrease physicochemical properties of Grim19 but has the same physiological activity to Grim19.

The said Grim19 protein preferably has the amino acid sequence represented by SEQ. ID. NO: 1.

In this invention, obesity indicates the status of excessive body fat. Particularly, when body fat is at least 25% by the total weight in a male or when body fat is at least 30% by the total weight in a female, it is defined as obesity. Clinically, when BMI (Body Mass Index) is at least 30.1, or when the current body weight exceeds the standard by 20% or more, it is defined as obesity. The cause of obesity includes genetic factors, environmental factors, and energy metabolism disorder, etc. Obesity can be divided according to the cause into simple obesity and symptomatic obesity. Simple obesity is caused by overeating and lack of exercise, and symptomatic obesity is caused by endocrine disorder, hypothalamic disorder, genetic disorder, frontal lobe disorder, and metabolic disorder.

The Grim19 protein of the present invention is excellent in inhibiting the differentiation of cytotoxic Th17 cells inducing the generation and secretion of inflammatory cytokines, and is excellent in suppressing abnormally activated immune cell functions and in activating Treg that can control inflammatory response. Grim19 is also able to regulate obesity related inflammatory environment by controlling STAT3, so that it can be effectively used for the prevention or treatment of not only simple obesity or symptomatic obesity but also obesity caused by immune dysregulation.

In obese patients, fat cells are enlarged and show different physical and physiological properties that can cause disorder in abdominal blood stream and lymphocyte flow, compared with those of normal weight people. The accompanied proliferation of giant cells induces the over-secretion of 50 kinds of inflammatory materials in fat cells, resulting in the complications including inflammation and metabolic syndrome. Obesity mediated diseases are exemplified by type II diabetes, osteoarthritis, non-alcoholic fatty liver disease, sleep apnea, pulmonary thromboembolism, hypertension, asthma, infertility, cancer, and depression. Obese people have the risk of getting diabetes twice as high as standard weight people, and hypertension 1.5 times as high as standard weight people. Severe obese people have the risk of getting diabetes 5 times as high as standard weight people, and the risk of hypertension 2.5 times as high as standard weight people. As obesity level goes severe, the risk of complications goes higher.

Once people becomes severe obese, their fat cells are enlarged 400 times as big as the normal size. This enlarged fat cell is not only big but also very hard and tight which is like touching tangerine, so that it is not easily broken. Standard fat cell is composed of 85% fat and 15% water, so when you press it with your finger, it is weak and crumbled. But, the enlarged fat cell of severe obese people has hard surface and is strong enough not to be broken. When fat cell is transformed like that, macrophages start to act. Basically, macrophages play a role in eliminating germs when they invade in our bodies. However, when fat cells grow abnormally big, macrophages consider it as an abnormal change, so that they keep growing to eat up the enlarged fat cells. As a result, macrophages become 400 times as big as the standard size, like the fat cells that have been grown 400 times as big as the standard, resulting in the giant cells.

The activity of macrophage is a kind of a normal clean system in human body. When germs enter our bodies, macrophages secrete inflammatory factors to eliminate the germs and to keep our body healthy. However, 400 times enlarged macrophages cannot play a role as a clean system. Make matter worse, the enlarged macrophages over-secret various inflammatory factors, which become a threat to a healthy body. Under the continued inflammation situation, blood vessels are enlarged with making wounds between them and making problems in blood circulation in many parts by fibrosis. In turn, unsmooth blood circulation makes macrophages secret more inflammatory factors. The additionally secreted inflammatory factors again generate unnecessary blood vessels to hinder blood circulation. Then, insulin malfunction is caused, resulting in diabetes. Hypertension is caused because of the narrowed blood vessel, caused by fibrosis, which makes blood circulation difficult and increase the pressure thereby. The complications of obesity depend where the enlarged macrophages induce inflammatory response. For example, when the giant cells induce inflammatory response in joint, arthritis is developed. When the giant cells induce inflammatory response in lung, pulmonary embolism is developed. If such inflammatory response is happening all over the body, immune system becomes weak. That is why the immune system of obese people is weaker than that of the standard weight people.

Therefore, the Grim19 of the present invention can be effectively used not only for the treatment of obesity but also for the treatment of obesity mediated inflammatory disease or obesity mediated auto-immune disease. So, the present invention can provide a pharmaceutical composition for the prevention or treatment of obesity mediated inflammatory disease or auto-immune disease.

The term “treatment” in this invention indicates the action to reverse or to relieve at least one of symptoms of the said disorder or disease targeted by the term or the action to inhibit or to prevent the progress of the disorder or disease, unless stated otherwise.

The pharmaceutical composition of the present invention for the prevention or treatment of obesity or lipid-related metabolic disease can contain a pharmaceutically effective dose of Grim19 protein alone or together with one or more pharmaceutically acceptable carriers, excipients, or diluents. The pharmaceutically effective dose mentioned above indicates the amount enough to prevent, improve, and treat obesity or lipid-related metabolic diseases.

The pharmaceutically effective dose of the Grim19 protein of the present invention is preferably 0.5˜100 mg/day/kg, and more preferably 0.5˜5 mg/day/kg. However, the pharmaceutically effective dose above can be adjusted according to the severity of obesity or lipid-related metabolic diseases, age, weight, health condition, and gender of patient, administration pathway, and treatment period, etc.

The said “pharmaceutically acceptable” herein indicates a composition that is acceptable physiologically and does not cause any allergic reaction or allergy like reaction such as gastrointestinal disorder and dizziness. The carriers, excipients and diluents are exemplified by lactose, dextrose, sucrose, sorbitol, mannitol, xylitol, erythritol, maltitol, starch, acacia rubber, alginate, gelatin, calcium phosphate, calcium silcate, cellulose, methyl cellulose, microcrystalline cellulose, polyvinyl pyrrolidone, water, methylhydroxybenzoate, propylhydroxybenzoate, talc, magnesium stearate and mineral oil. In addition, fillers, antiaggregations, lubricants, wetting agents, flavors, emulsifiers, and antiseptics, can also be included.

The composition of the present invention can be formulated properly in a proper form by those in the art, in order for the active ingredient to be delivered fast, continuously, or delayed purposefully after administered to mammals. The composition can be formulated in the forms of powders, granules, tablets, emulsions, syrups, aerosols, soft or hard gelatin capsules, sterilized injections, or sterilized powders.

The pharmaceutical composition of the present invention for the prevention or treatment of obesity or lipid-related metabolic disease can be administered via various pathways including oral administration, transdermal, hypodermic, intravenous, and intramuscular injection. The dose of the active ingredient can be adjusted according to the administration pathway, age, gender, and weight of patient, severity of disease, etc. The composition for the prevention or treatment of obesity or lipid-related disease of the invention can be co-administered with any informed compound that is known to have the effect of preventing, improving, or treating obesity or lipid-related metabolic diseases.

The present invention also provides a pharmaceutical composition for the prevention or treatment of obesity of lipid-related metabolic diseases comprising the Grim19 protein of the invention or the polynucleotide encoding the same as an active ingredient.

The polynucleotide encoding the Grim19 protein can include DNA or RNA, and more preferably DNA represented by SEQ. ID. NO: 2.

The said polynucleotide can be introduced in an expression vector such as a plasmid or a virus vector by the conventional method and then the expression vector can be introduced in a target cell via infection or transduction, etc.

The plasmid expression vector is a way of delivering plasmid DNA directly into a human cell via a human applicable gene delivery method approved by FDA (Nabel, E. G., et al., Science, 249:1285-1288, 1990). The plasmid DNA can be evenly purified, unlike the virus vector. The plasmid expression vector usable in this invention is one of the informed mammal expression plasmid well known to those in the art, which is exemplified by pRK5 (European Patent No. 307,247), pSV168 (International Patent Publication No. 91/08291), and pVL1392 (PharMingen), but not always limited thereto.

The plasmid expression vector containing the polynucleotide of the present invention can be introduced in cells by one of the informed methods well-known to those in the art which is selected from the group consisting of transient transfection, microinjection, transduction, cell fusion, calcium phosphate precipitation, liposome-mediated transfection, DEAE dextran-mediated transfection, polybrene-mediated transfection, electroporation, gene gun, and other well known methods for inserting DNA in cells (Wu et al., J. Bio. Chem., 267:963-967, 1992; Wu and Wu, J. Bio. Chem., 263:14621-14624, 1988), but not always limited thereto.

The Grim19 expression vector can be administered by an informed method, which is exemplified by local administration, parenteral administration, oral administration, intranasal administration, intravenous administration, intramuscular administration, subcutaneous administration, or other proper pathways.

The present invention provides a pharmaceutical composition comprising an expression vector harboring the Grim19 protein or the polynucleotide encoding the same as an active ingredient for the prevention or treatment of obesity or lipid-related metabolic diseases. In addition, the present invention provides a composition comprising an expression vector harboring the Grim19 protein or the polynucleotide encoding the same as an active ingredient for the inhibition of obesity or lipid-related metabolic diseases.

The present invention also provides a method for screening a therapeutic agent for obesity or lipid-related metabolic diseases comprising the steps of (a) culturing the Grim19 protein or the recombinant cells expressing the protein together with a candidate material; and (b) measuring the effect of the candidate material on the activity or the intracellular level of Grim19 protein. At this time, the activity or the intracellular level of Grim19 protein can be measured by coimmunoprecipitation, RIA, ELISA, immunohistochemical method, Western blotting, or FACS.

The candidate material for the therapeutic agent for the treatment of obesity or lipid-related metabolic diseases of the invention can be a material that is able to increase the activity or the intracellular level of Grim19 as the above and the up-regulation of intracellular level of Grim19 indicates the up-regulation of Grim19 expression itself or the inhibition of Grim19 degradation resulting in the up-regulation of Grim19.

### Advantageous Effect

The Grim19 protein of the present invention was confirmed to be excellent in reducing fat cells and total cholesterol content, and also excellent in inhibiting the differentiation of cytotoxic Th17 cells inducing the generation and secretion of inflammatory cytokines. The Grim19 protein was also able to control obesity related inflammatory environment, that is it could regulate STAT3 mediated inflammatory response. Therefore, the Grim19 of the present invention can be effectively used in the preparation of a therapeutic agent and functional food for treating obesity or lipid-related metabolic diseases effectively.

### DESCRIPTION OF DRAWINGS

The application of the preferred embodiments of the present invention is best understood with reference to the accompanying drawings, wherein:

FIG. 1 presents the weights of the Grim19 TG mouse group fed with 60 kcal high fat diet, the control mouse C57BL/6 (H-2kb) group fed with 60 kcal high fat diet, and the group fed with 16 kcal general diet, measured for 10 weeks.

FIG. 2a and FIG. 2b illustrate the results of the observation by the naked eye of the liver and body of the animals sacrificed on day 61 and the measurement of their weights according to a preferred embodiment of the invention.

FIG. 3 illustrates the results of hematoxylin-eosin staining and oil red O staining with the frozen section (7 μm) of the liver of the mouse sacrificed according to a preferred embodiment of the invention.

FIG. 4a and FIG. 4b illustrate the shape and the weight of fat between the retroperitoneal region and the interscapular region of the animal sacrificed according to a preferred embodiment of the invention.

FIG. 5 presents the weights of both white fat and brown fat in the interscapular area of the control group fed with normal diet and the Grim19 TG mouse group measured according to a preferred embodiment of the invention.

FIG. 6a and FIG. 6b are graphs illustrating the levels of glucose, triglyceride, total cholesterol, HDL-cholesterol, LDL-cholesterol, AST, and ALT of three experimental animal groups measured according to a preferred embodiment of the present invention.

FIG. 7a illustrates the results of FACS with the inflammatory cytokine in the spleen cells of the Grim19 TG group, the high fat diet WT mouse group, and the normal WT mouse group.

FIG. 7b˜FIG. 7f are photographs and graphs illustrating the results of confocal histostaining with the spleen cells of the Grim19 TG group, the high fat diet WT mouse group, and the normal WT mouse group.

FIG. 7g illustrates the results of Western blotting confirming the phosphorylation of STAT3 mediated by Grim19, performed according to a preferred embodiment of the present invention.

FIG. 8 is an electron micrograph illustrating the effect of Grim19 on the fat cells in the liver tissue of the high fat diet mouse group.

FIG. 9 is a photograph illustrating the results of hematoxylin-eosin staining performed to investigate the effect of Grim19 on the fat cells in the liver tissue of the high fat diet mouse group, in which HFD indicates high fat diet.

FIG. 10a is a photograph illustrating the results of oil red O staining performed to investigate the effect of Grim19 on the differentiation of fat cells.

FIG. 10b˜FIG. 10e are graphs illustrating the expressions of white fat and brown fat related genes in the cells transfected with Grim19.

FIG. 11a and FIG. 11b are graphs illustrating the expression changes of brown fat gene of the mouse transformed with Grim19 under the low temperature stress condition.

FIG. 12 is a set of a graph and a photograph of stained human macrophages illustrating the inhibition of hyperlipidemia by Grim19.

### BEST MODE

Practical and presently preferred embodiments of the present invention are illustrative as shown in the following Examples. However, it will be appreciated that those skilled in the art, on consideration of this disclosure, may make modifications and improvements within the spirit and scope of the present invention.

### EXAMPLE 1

### Investigation of Weight Reducing Effect of Grim19

To investigate whether or not Grim19 was effective in treating obesity, the present inventors first constructed Grim19 over-expression vector to over-express Grim19 protein. Grim19 cDNA was synthesized with substitution of codon via codon optimization in order to be well expressed in mammals (Table 1) (TOPgenetech, Canada), which was then digested with BamH1 and Xho1, followed by insertion in pcDNA3.1+ (Promega). As a result, the recombinant vector pcDNA3.1+Grim19 with Grim19 cDNA inserted was constructed. E. coli was transformed with the recombinant vector and then the plasmid DNA was obtained by using a plasmid extraction kit (Qiagen).

NIH3T3 cells were transfected with the Grim19 over-expression vector, followed by Western blotting to confirm the over-expression of Grim19. The Grim19 over-expression vector was handed over to Macrogen to put the order of TG mouse construction. TG mouse construction performed by Macrogen was composed of the following steps: preparing an expression vector by inserting Grim19 cDNA in TG vector; collecting zygotes from the oviduct of a female mouse induced with super ovulation by PMSG and hCG hormone injection; performing microinjection of Grim19 DNA solution under the microscope; selecting survived zygotes; and transplanting the survived zygotes into the oviduct of a surrogate mother mouse, followed by delivery. 2 weeks after the birth, the tail of the new-born mouse was cut, from which genomic DNA was extracted to investigate whether or not the gene was successfully inserted. PCR was performed with the gene to confirm germline transmission, resulting in the construction of the transgenic Grim19 mouse.

The transgenic Grim19 TG mouse and the normal mouse (C57BL/6(H-2kb)) were fed with 60 kcal high fat diet. Another normal mouse group (C57BL/6(H-2kb)) was fed with 16 kcal standard diet. These three mouse groups were used for the following experiment.

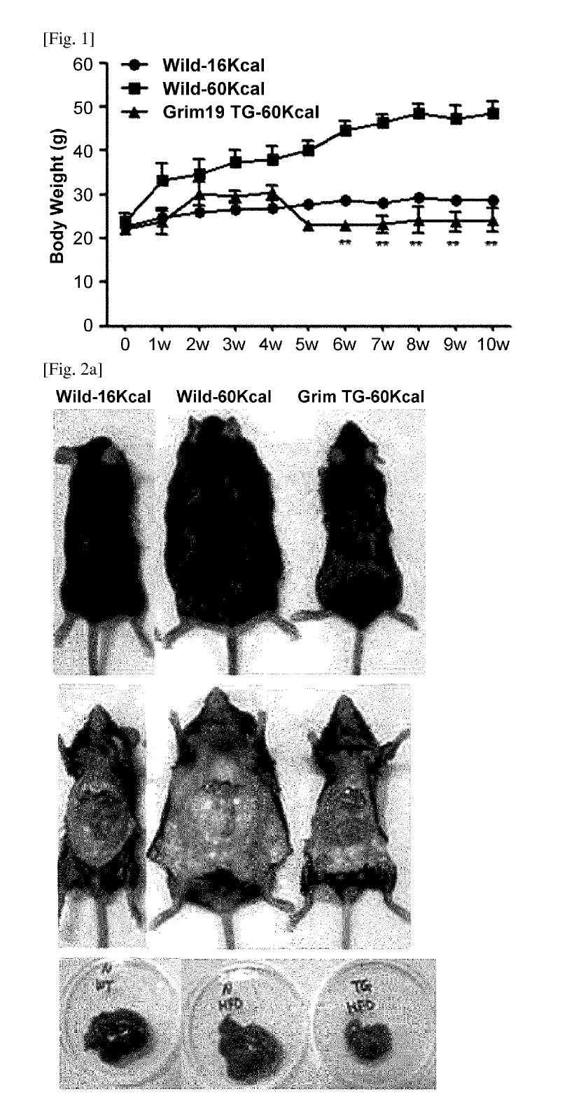

Body weights of those three experimental groups were measured for 10 weeks. As a result, the weight of the normal mouse group fed with high fat diet was gradually increased to obesity level, compared with that of the normal mouse fed with standard diet, while the weight of the Grim19 TG mouse group transformed to express Grim19 protein was not increased even after fed with high fat diet but was maintained the standard weight similar to that of the normal mouse group fed with standard diet (FIG. 1).

The above results indicate that the Grim19 of the present invention has the activity of inhibiting weight gaining, so that it is effective in inhibiting obesity.

TABLE 1
Grim19 sequence

| | |
|---|---|
| Grim19 | MAASKVKQDMPPPGGYGPIDYKRNLPRRGLSGYSMFAV |
| protein | GIGALIFGYWRMMRWNQERRRLLIEDLEARIALMPLFQ |
| sequence | AEKDRRTLQILRENLEEEAIIMKDVPNWKVGESVFHTT |
| (SEQ. ID. | RWVPPLIGEMYGLRTKEEMSNANFGFTWYT |
| NO: 1) | |
| Grim19 | GGATCCAGTATGGCGGCGTCGAAGGTGAAGCAGGACAT |
| cDNA | GCCCCCGCCAGGGGGCTACGGCCCCATCGACTACAAGC |
| sequence | GGAACCTGCCCCGCCGGGGACTGTCGGGGTACAGCATG |
| (SEQ. ID. | TTTGCTGTGGGCATCGGGGCCTTGATCTTTGGCTACTG |
| NO: 2) | GAGAATGATGAGGTGGAACCAGGAGCGCAGGCGCCTGC |
| | TGATTGAGGACTTGGAGGCCAGGATCGCCCTCATGCCG |
| | CTCTTCCAGGCAGAGAAGGACCGGAGGACCCTGCAGAT |
| | TCTCCGGGAAAACCTGGAGGAGGAAGCCATCATCATGA |
| | AGGATGTGCCCAACTGGAAGGTGGGCGAGTCTGTGTTC |
| | CATACCACACGATGGGTGCCACCCCTCATTGGCGAGAT |
| | GTATGGGTTGCGCACCAAGGAGGAGATGAGCAATGCCA |
| | ACTTCGGCTTCACCTGGTACACTTAGGGCCTCGAG |
| Codon | GGATCCAGTATGGCTGCCAGCAAGGTGAAGCAGGACAT |
| optimized | GCCCCCTCCTGGCGGCTACGGCCCCATCGACTACAAGA |
| Grim19 | GAAACCTGCCCAGAAGAGGCCTGAGCGGCTACAGCATG |
| (SEQ. ID. | TTCGCCGTGGGCATCGGCGCCCTGATCTTCGGCTACTG |
| NO: 3) | GAGAATGATGAGATGGAACCAGGAGAGACGGAGACTGC |
| | TGATCGAGGACCTGGAGGCCAGAATCGCCCTGATGCCC |
| | CTGTTCCAGGCCGAGAAGGACAGAAGAACCCTGCAGAT |
| | CCTGAGAGAGAACCTGGAGGAGGAGGCCATCATCATGA |
| | AGGACGTGCCCAACTGGAAGGTGGGCGAGAGCGTGTTC |
| | CACACCACAAGATGGGTGCCTCCCCTGATCGGCGAGAT |
| | GTACGGCCTGAGAACCAAGGAGGAGATGAGCAACGCCA |
| | ACTTCGGCTTCACCTGGTACACCTAGGGACTCGAG |

### EXAMPLE 2

### Anti-Obesity Effect of Grim19 Confirmed by Histological Analysis

### <2-1> Comparison of Weights and Tissue Sizes by Macroscopy

Each mouse group was sacrificed on Day 61 which is the time point that showed a significant difference in obesity level among the three experimental group animals prepared in Example 1, and the size of the body and the liver of each sacrificed mouse was observed with the naked eye.

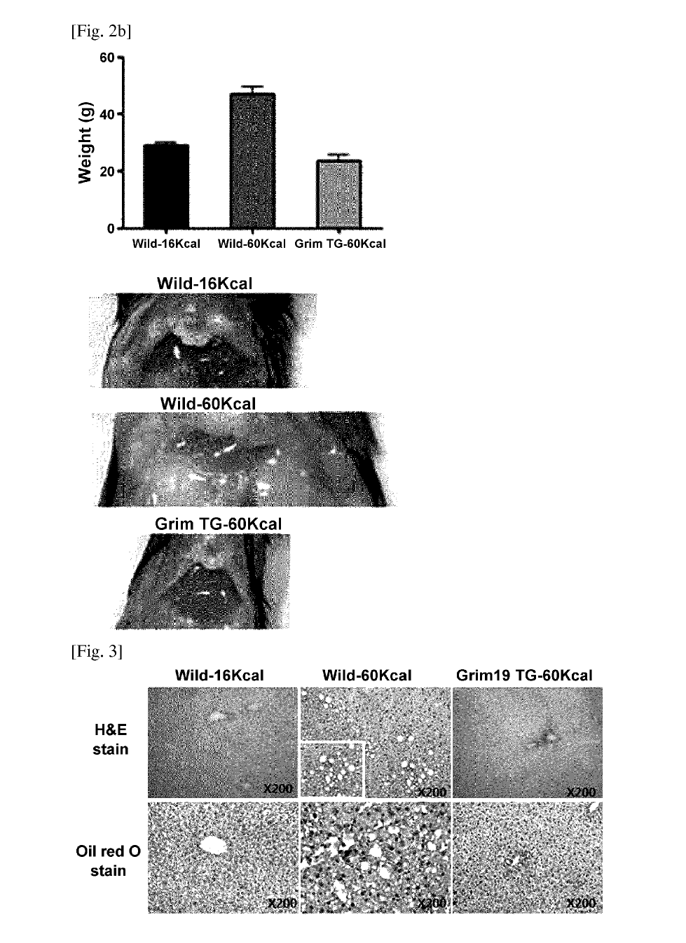

As a result, as shown in FIG. 2a and FIG. 2b, the body size of the mouse fed with high fat diet was approximately 1.5 times bigger than that of the mouse fed with standard diet. The liver of the mouse fed with high fat diet was yellow because of the fat accumulation. However, the body size of the Grim19 TG mouse group fed with high fat diet was just similar to that of the normal mouse fed with standard diet and the liver of the Grim19 TG mouse showed similar shape and color to that of the normal diet mouse.

### <2-2> Immunohistochemical Staining

Liver tissue was extracted from each mouse group of Example <2-1> to make frozen sections (7 μm). The frozen sections were stained with hematoxylin-eosin and oil red O, followed by observation of fat cells.

As a result, the number of fat cells was certainly increased and the size thereof was also enlarged in the mouse group fed with high fat diet, compared with those of the mouse group fed with standard diet (normal mouse group). In the meantime, the number and the size of fat cells of the Grim19 TG mouse group fed with high fat diet were similar to those of the normal mouse group (FIG. 3).

### <2-3> Observation of FAT PAD Over the Area and Measurement of the Weight

The fat in-between the retrooperitoneal region and the interscapular region was collected from each mouse group of Example <2-1> and the shape and the size of the fat were observed.

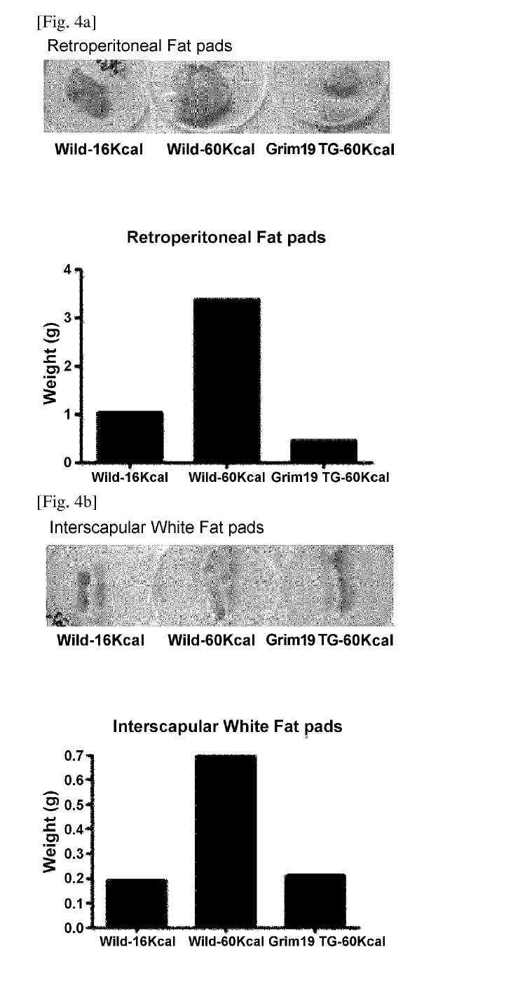

As a result, as shown in FIG. 4a and FIG. 4b, the fat in the area between the retroperitoneal region and the interscapular region of the mouse group fed with high fat diet was at least 3 times increased, compared with that of the normal mouse group fed with standard diet. In the meantime, FAT PAD of the Grim19 TG mouse group fed with high fat diet was similar to that of the normal diet mouse group.

Therefore, it was confirmed by histological observation and analysis that the Grim19 of the present invention was effective in inhibiting obesity caused by high fat diet.

### EXAMPLE 3

### Comparative Analysis of Brown Fat and White Fat of Grim19 TG Mouse

After confirming through the above experiments that the Grim19 of the present invention was effective in preventing and inhibiting obesity, the present inventors further performed the following experiment to investigate how Grim19 could affect the formation of white fat and brown fat.

White fat is responsible for changing excessive energy into triglyceride and deposit thereof. Therefore, when energy is over-generated by lack of exercise or overeating, the number of white fat cells increases, resulting in the obesity and changing the physical constitution to be apt to gain weight easily. In the meantime, brown fat plays a role in consuming the deposited energy by generating heat with that, so it is not responsible for the weight gaining and obesity.

To investigate the effect of Grim19 on white fat and brown fat, the present inventors raised mice, precisely the Grim19 TG mice fed with standard diet and the normal mice that was not transformed and fed with standard diet, for 10 weeks. Then, the mice were sacrificed, followed by autopsy. The mass and the accumulation of brown fat and white fat were analyzed.

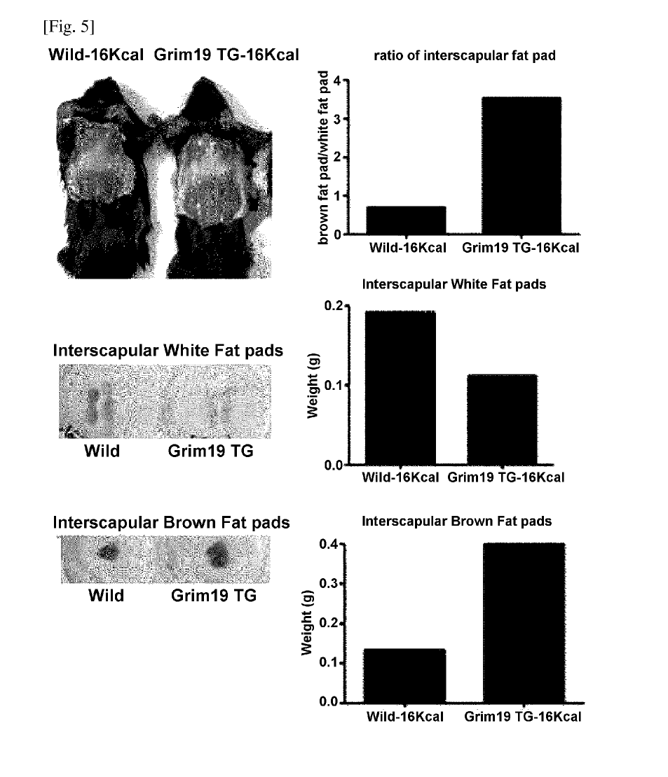

White fat and brown fat in the interscapular region of both the normal control group and the Grim19 TG mouse group were separated and weighed. As a result, white fat of the Grim19 TG mouse group was reduced, compared with that of the normal mouse group. In the meantime, brown fat of the Grim19 TG mouse group was increased, compared with that of the normal mouse group (FIG. 5).

The ratio of white fat to brown fat was also investigated. As a result, in the Grim19 over-expressing in vivo mouse model, whit fat formation was reduced and the reduced number of white fat cells was converted into brown fat cells.

### EXAMPLE 4

### Analysis of Preventive or Treating Effect of Grim19 on Lipid-Related Metabolic Diseases

To investigate whether or not the Grim19 of the present invention could be effective in preventing or treating lipid-related metabolic diseases, the present inventors first measured the blood glucose, triglyceride, cholesterol, and free fatty acid of each mouse group of Example <2-1>. That is, each mouse group of Example <2-1> was anesthetized with isoflurane, and then blood was collected from the heart. The blood was loaded in a centrifuge tube, followed by centrifugation at 3000 rpm for 20 minutes to separate serum. The obtained serum was frozen and stored at −70° C. until used for analysis.

The levels of glucose, triglyceride, total cholesterol, HDL-cholesterol, LDL-cholesterol, AST, and ALT in serum were analyzed by an automatic analyzer (Kuadro, Italy).

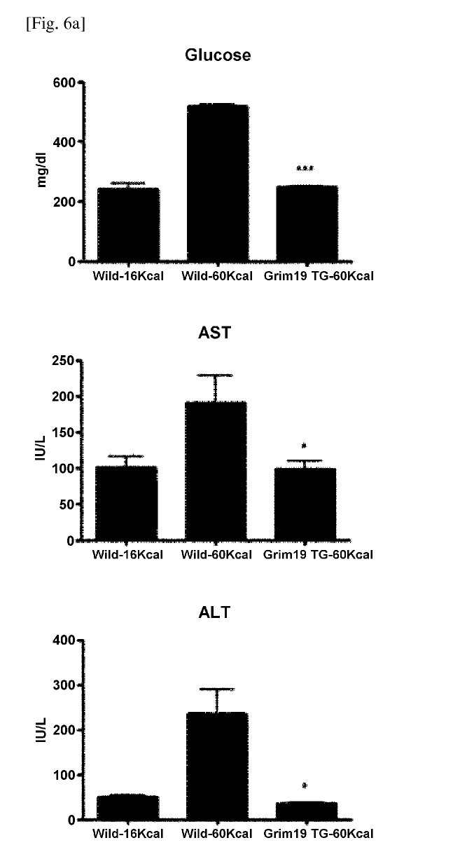

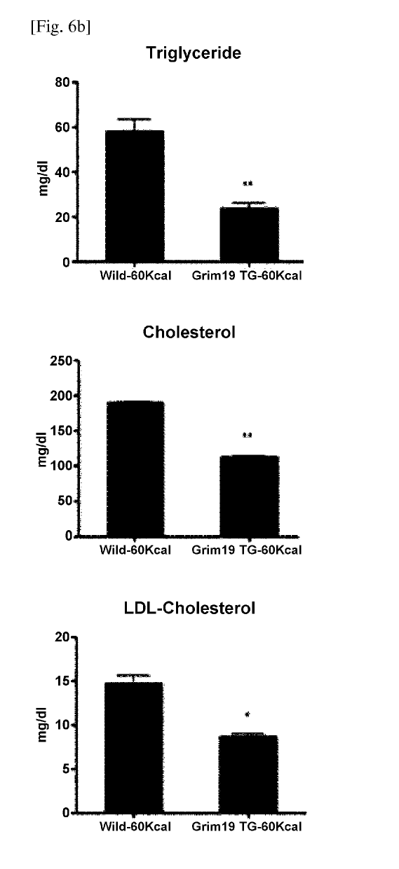

As a result, unlike other obesity induced mouse models, the Grim19 TG mouse group fed with high fat diet demonstrated significantly reduced levels of glucose, triglyceride, total cholesterol, HDL-cholesterol, LDL-cholesterol, AST, and ALT in serum. The levels of the above components in the Grim19 TG mouse group fed with high fat diet were almost similar to those of the normal mouse group fed with standard diet (FIG. 6a and FIG. 6b).

From the above results, it was confirmed that the Grim19 of the present invention was excellent in reducing the levels of glucose, triglyceride, cholesterol, AST, and ALT, which had been increased by obesity, and therefore Grim19 was confirmed to be effectively used for the prevention and treatment of obesity and hyperlipidemia caused by high cholesterol and fat accumulation and other metabolic diseases caused by obesity and lipid metabolic dysregulation.

### EXAMPLE 5

### Regulation of T Helper Cell and STAT3 by Grim19

To investigate the correlation between obesity and inflammatory response, the present inventors performed FACS to observe inflammatory cytokines in spleen cells of the normal and the Grim19 TG mouse group both fed with high fat diet. As a control, WT mouse fed with standard diet was prepared.

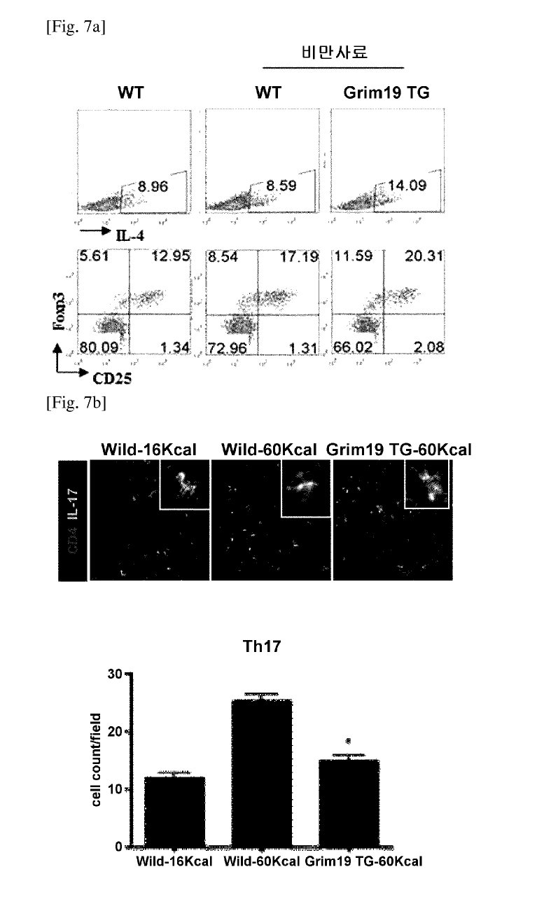

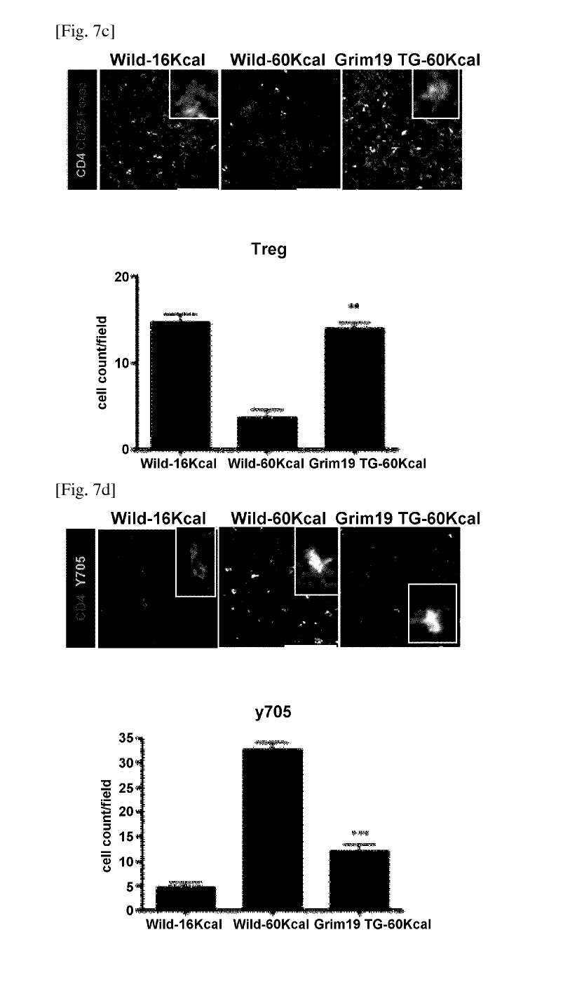

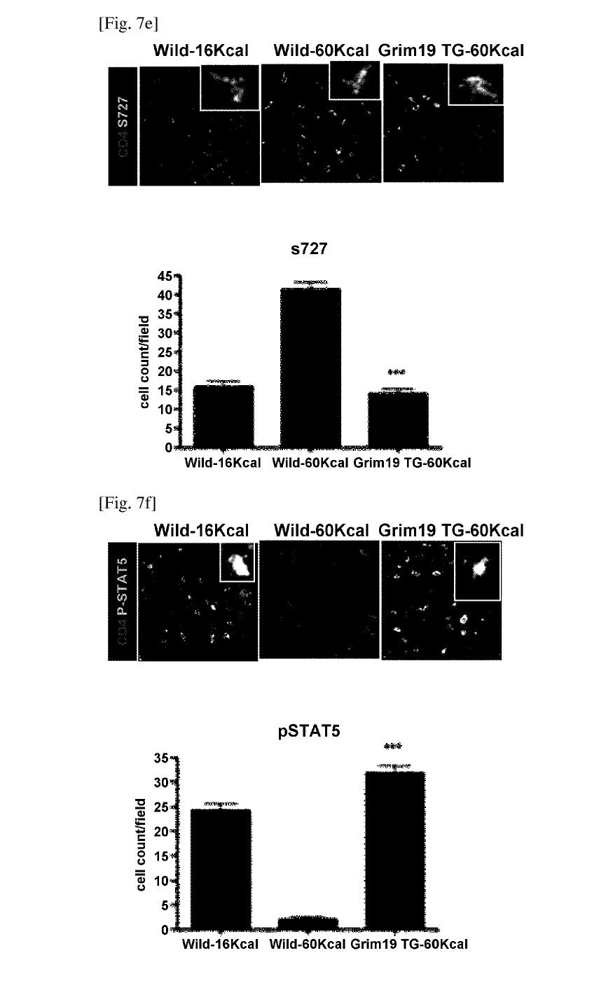

As a result, T helper cells (Th2) and Treg cells involved in anti-inflammatory response were significantly increased in the Grim19 TG mouse group (FIG. 7a).

Spleen confocal histostaining also confirmed that the Grim19 of the present invention suppressed Th17 cells involved in inflammatory response and inhibited the expressions of p-STAT 727 and 705, but promoted or increased the activity of regulatory T cells (Treg) and the expression of p-STATS (FIGS. 7b˜7f).

The Grim19 protein of the present invention is the molecule that is able to inhibit STAT3, particularly known to be involved in the regulation of p-STAT3 727. The present inventors first observed the activity of STAT3 in the Grim19 TG mouse group. As a result, it was observed that p-STAT3 was significantly suppressed in the Grim19 TG mouse group, compared with that of the control WT mouse group. This result was consistent with that of the Grim19 TG mouse group fed with high fat diet (FIG. 7g).

Therefore, it is believed that the Grim19 protein of the present invention can regulate inflammatory response by mediating STAT-3 in the obesity related inflammation condition.

In conclusion, the above results indicate that the Grim19 protein of the present invention is excellent in reducing fat cells and total cholesterol, so that Grim19 can be used for the prevention and treatment of obesity and lipid-related metabolic diseases including hyperlipidemia by such a pharmacological mechanism of Grim19.

Further, Grim19 is also excellent in inhibiting the differentiation of cytotoxic Th17 cells inducing the generation and secretion of inflammatory cytokines and can regulate inflammatory response by mediating STAT3 in the obesity related inflammation condition, suggesting that Grim19 can be used for the prevention and treatment of obesity.

### EXAMPLE 6

### Effect of Grim19 on Fat Cells in Liver Tissue

To investigate the effect of Grim19 on fat cells in liver tissue of the mouse group fed with high fat diet, the present inventors obtained liver tissue from the Grim19 TG mouse group having C57BL/6 (H-2kb) background that had been fed with 60 kcal high fat diet, the Grim19 TG mouse group fed with 16 kcal standard diet, the control mouse group C57BL/6 (H-2kb) fed with 60 kcal high fat diet, and the mouse group fed with 16 kcal standard diet, followed by observation under electron microscope. As a result, the size of the fat in the liver of the

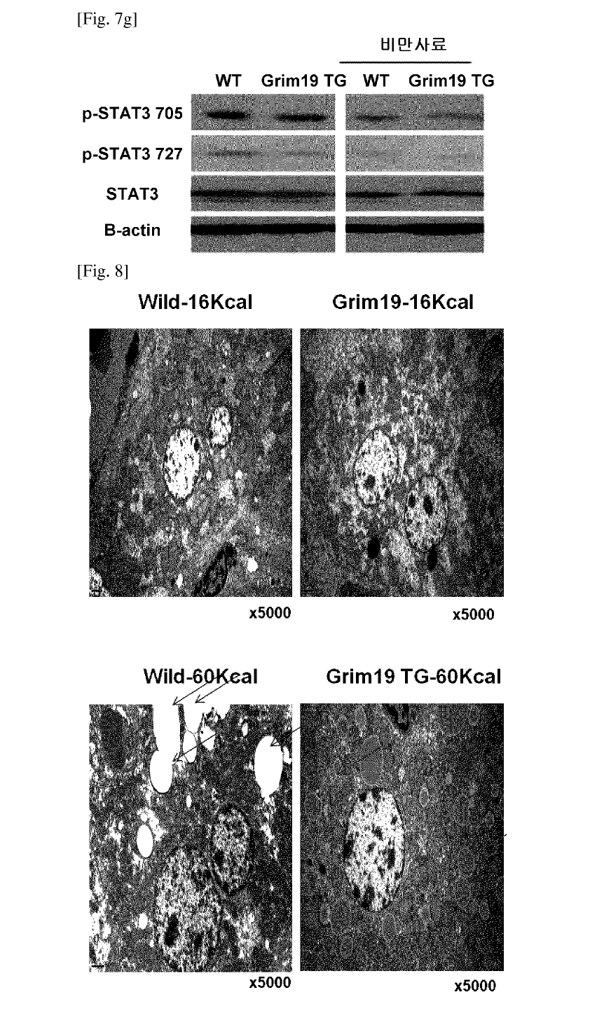

Grim19 TG mouse group was reduced, compared with that of the C57BL/6 mouse group fed with high fat diet (FIG. 8).

### EXAMPLE 7

### Effect of Grim19 on Fat Cells in Abdominal Fat

To investigate the effect of Grim19 on fat cells in abdominal fat of the mouse group fed with high fat diet, abdominal fat was extracted from the Grim19 TG mouse group having C57BL/6 (H-2kb) background that had been fed with 60 kcal high fat diet, the Grim19 TG mouse group fed with 16 kcal standard diet, the control mouse group C57BL/6 (H-2kb) fed with 60 kcal high fat diet, and the mouse group fed with 16 kcal standard diet. The obtained abdominal fat was made into paraffin blocks. The paraffin blocks were sliced into 7 μm thick sections, followed by staining with hematoxylin-eosin by the same manner as described in Example <2-2>.

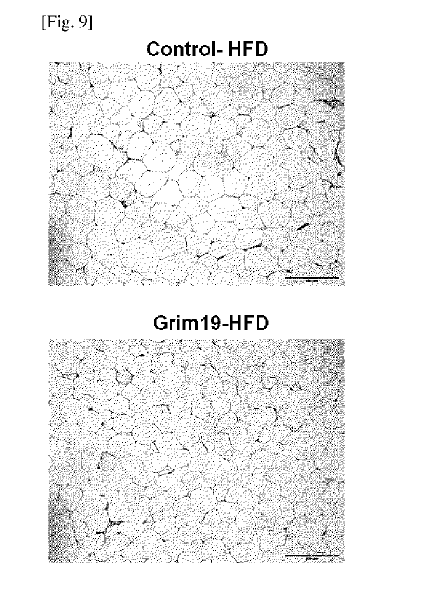

As a result, the size of abdominal fat of the Grim19 mouse fed with high fat diet was reduced, compared with that of the C57BL/6 mouse fed with high fat diet (FIG. 9).

### EXAMPLE 8

### Effect of Grim19 on the Differentiation of Fat Cells

3T3 pre-adipocytes were transfected with Grim19 to over-express Grim19 gene there and fat cell differentiation was induced thereafter. On the same day of the transfection, the medium was replaced with 3T3-L1 differentiation medium (DM-2L1, Zenbio), followed by culture for 3 days. The medium was replaced again with 3T3-L1 adipocyte Medium (AM-1-L1, Zenbio). Then, the differentiation of fat cells was observed. As for control, pre-adipocytes transfected with pcDNA3.1 were prepared. Oil red O staining was performed by the same manner as described in Example <2-2> to observe the differentiation of fat cells. Particularly, cells cultured in a 24-well plate were stained as they were by oil red O staining. The cells were dipped in 10% formalin for 30 minutes, followed by washing with deionized water. Then, the cells were treated with 60% isopropanol and reacted with oil red O for 5 minutes. The cells were washed with deionized water, followed by counter-staining with hematoxylin. The cells were then observed under microscope.

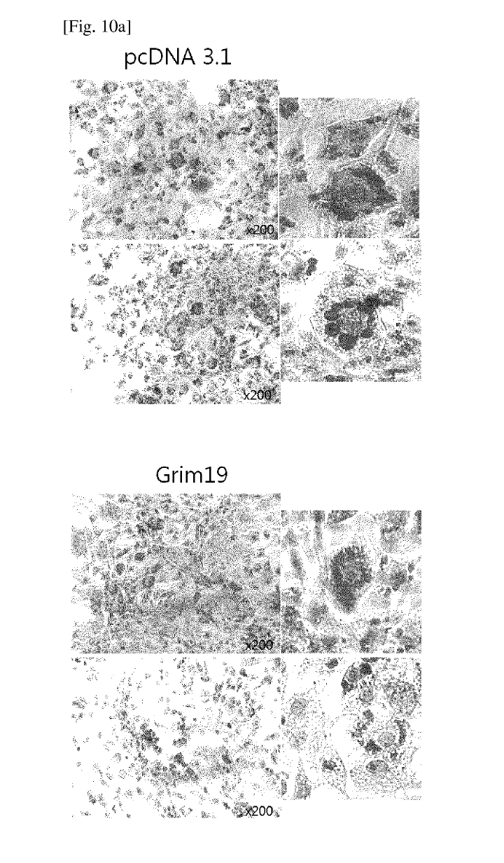

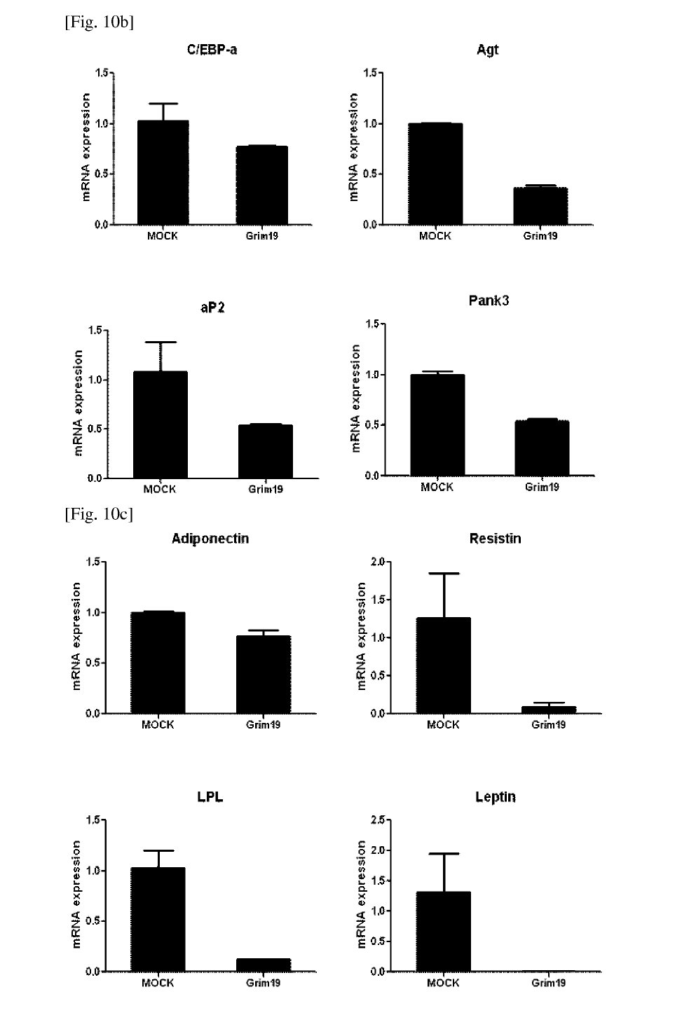

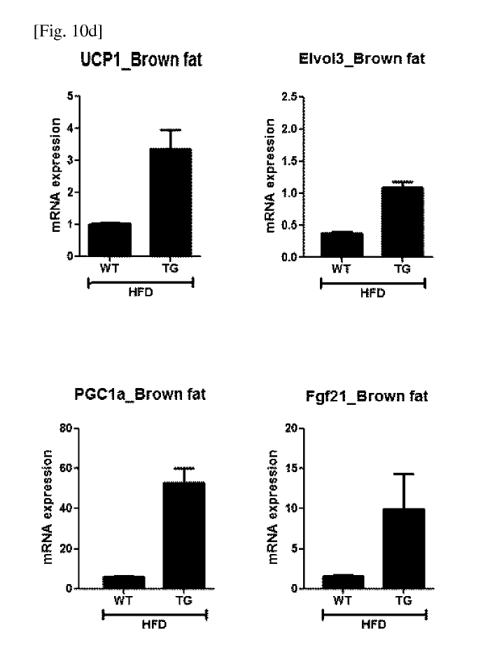

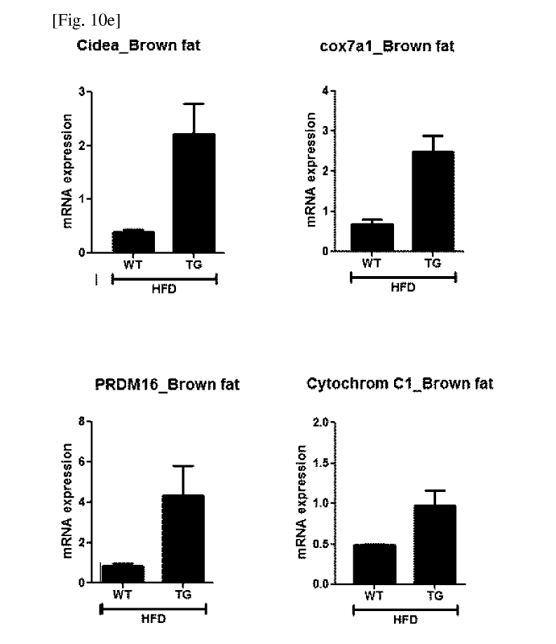

As a result, oil red O positive cells were reduced in those cells transfected with Grim19, suggesting that Grim19 could inhibit the differentiation of fat cells (FIG. 10a).

After synthesizing cDNA from RNA extracted from the differentiated fat cells, the gene expressed in white fat was investigated by real-time PCR. RT-PCR was performed with LightCycler FastStart DNAmaster SYBR green I (Takara) using ABI PCR machine. The reaction mixture for PCR was prepared by mixing 1 μl of 1 μl cDNA diluted at the ratio of 1:3, 10 μl of LightCycler FastStart DNAmaster SYBR green I (Takara), and 1 μl of Taqman probe (Applied Biosystems), to which distilled water was added to make the total volume 20 μl. The reaction condition was set as 95° C. for 10 seconds and 60° C. for 30 seconds (50 cycles). Cycle threshold (Ct) value was analyzed and the relative expressions of mRNAs of C/EBP-a, Agt, aP2, Pank3, Adiponectin, Resistin, LPL, and Leptin were calculated by comparing the mRNA expression of β-actin, the house keeping gene.

The primers used for the above RT-PCR were C/EBP-a (Forward: 5′-CAA GAA CAG CAA CGA GTA CCG-3′ (SEQ. ID. NO: 4), Reverse: 5′-GTC ACT GGT CAA CTC CAG CAC-3′, (SEQ. ID. NO: 5)), Agt (Forward: 5′-GCA CCC TGG TCT CTT TCT ACC-3′ (SEQ. ID. NO: 6), Reverse: 5′-TGT GTC CAT CTA GTC GGG AGG-3′ (SEQ. ID. NO: 7)), aP2 (Forward: 5′-GAT GCC TTT GTG GGA ACC T-3′ (SEQ. ID. NO: 8), Reverse: 5′-CTG TCG TCT GCG GTG ATT T-3′ (SEQ. ID. NO: 9)), PANK3 (Forward: 5′-TGC TCT AGT GTC CCA TTT CTG CCT-3′ (SEQ. ID. NO: 10), Reverse: 5′-AGC TGG AAC AGC AAC TAG GAA-3′ (SEQ. ID. NO: 11)), Adiponectin (Forward: 5′-GTC AGT GGA TCT GAC GAC ACC AA-3′ (SEQ. ID. NO: 12), Reverse: 5′-ATG CCT GCC ATC CAA CCT G-3′ (SEQ. ID. NO: 13)), Resistin (Forward: 5′-AAG AAC CTT TCA TTT CCC CTC CT-3′ (SEQ. ID. NO: 14), Reverse: 5′-GTC CAG CAA TTT AAG CCA ATG TT-3′ (SEQ. ID. NO: 15)), LPL (Forward: 5′-GGA AGA GAT TTC TCA GAC ATC G-3′ (SEQ. ID. NO: 16), Reverse: 5′-CTA CAA TGA CAT TGG AGT CAG G-3′ (SEQ. ID. NO: 17)), and Leptin (Forward: 5′-CCT CAT CAA GAC CAT TGT CAC C-3′ (SEQ. ID. NO: 18), Reverse: 5′-TCT CCA GGT CAT TGG CTA TCT G-3′ (SEQ. ID. NO: 19)). As a result, the expression of white fat gene was all reduced, compared with the control (FIG. 10b and FIG. 10c).

After synthesizing cDNA from RNA extracted from the differentiated fat cells, the gene expressed in brown fat was investigated by real-time PCR. RT-PCR was performed with LightCycler FastStart DNAmaster SYBR green I (Takara) using ABI PCR machine. The reaction mixture for PCR was prepared by mixing 1 μl of 1 μl cDNA diluted at the ratio of 1:3, 10 μl of LightCycler FastStart DNAmaster SYBR green I (Takara), and 1 μl of Taqman probe (Applied Biosystems), to which distilled water was added to make the total volume 20 μl. The reaction condition was set as 95° C. for 10 seconds and 60° C. for 30 seconds (50 cycles). Cycle threshold (Ct) value was analyzed and the relative expressions of mRNAs of UCP1, Elvol3, PGC1a, Fgf21, Cidea, Cox7a1, PRDM16, and Cytochrom C1 were calculated by comparing the mRNA expression of β-actin, the house keeping gene.

The primers used for the above RT-PCR were UCP1 (Forward: 5′-CTT TGC CTC ACT CAG GAT TGG-3′ (SEQ. ID. NO: 20), Reverse: 5′-ACT GCC ACA CCT CCA GTC ATT-3′ (SEQ. ID. NO: 21)), Elvol3 (Forward: 5′-CGG GTT AAA AAT GGA CCT GA-3′ (SEQ. ID. NO: 22), Reverse: 5′-CCA ACA ACG ATG AGC AAC AG-3′ (SEQ. ID. NO: 23)), PGC1a (Forward: 5′-GTC AAC AGC AAA AGC CAC AA-3′ (SEQ. ID. NO: 24), Reverse: 5′-TCT GGG GTC AGA GGA AGA GA-3′ (SEQ. ID. NO: 25)), FGF21 (Forward: 5′-CCT CTA GGT TTC TTT GCC AAC AG-3′ (SEQ. ID. NO: 26), Reverse: 5′-AAG CTG CAG GCC TCA GGA T-3′ (SEQ. ID. NO: 27)), Cidea (Forward: 5′-GCC GTG TTA AGG ATT CTG CTG-3′ (SEQ. ID. NO: 28), Reverse: 5′-TGC TCT TCT GTA TCG CCC AGT-3′ (SEQ. ID. NO: 29)), Cox7a1 (Forward: 5′-AGA AAA CCG TGT GGC AGA GA-3′ (SEQ. ID. NO: 30), Reverse: 5′-CAG CGT CAT GGT CAG TCT GT-3′ (SEQ. ID. NO: 31)), PRDM16 (Forward: 5′-GAC ATT CCA ATC CCA CCA GA-3′ (SEQ. ID. NO: 32), Reverse: 5′-CAC CTC TGT ATC CGT CAG CA-3′ (SEQ. ID. NO: 33)), and Cytochrom C1 (Forward: 5′-GCT ACC CAT GGT CTC ATG GT-3′ (SEQ. ID. NO: 34), Reverse: 5′-CAT CAT CAT TAG GGC CAT CC-3′ (SEQ. ID. NO: 35)). As a result, it was confirmed that the expression of brown fat gene in the cells transfected with Grim19 was increased, compared with that of the control, suggesting that Grim19 could induce brown fat (FIG. 10d and FIG. 10e).

### EXAMPLE 9

### Changes of Brown Fat Gene Under Stress Condition

The Grim19 TG mouse group and the control C57BL/6 mouse group were fed with standard diet and high fat diet for 11 weeks. Then, the animals were sacrificed after exposing on the temperature of 4° C. for 15 hours. RNA was extracted from abdominal fat, from which cDNA was synthesized. Real-time PCR was performed by the same manner as described in Example 8 to investigate the gene expressed in brown fat.

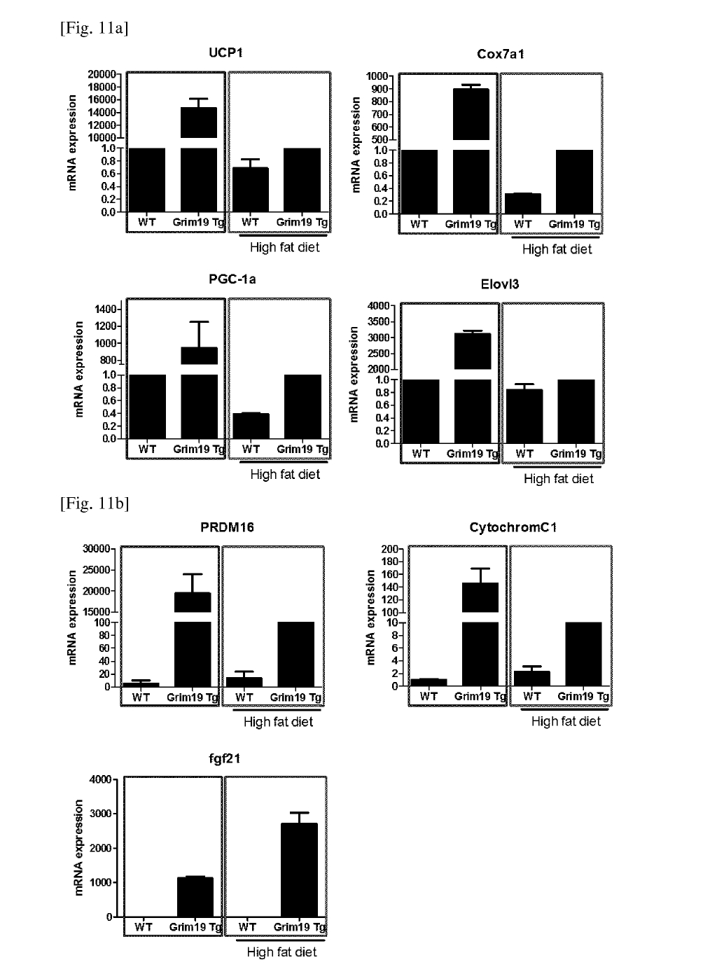

As a result, it was confirmed that brown fat gene was increased, compared with the control (FIG. 11a and FIG. 11b). This result indicates that brown fat gene expression is promoted in the presence of Grim19 if the environmental condition fits for the generation of brown fat.

### EXAMPLE 10

### Anti-Hyperlipidemia Effect of Grim19

Arteriosclerosis is progressed when macrophages are changed into foam cells by lipid oxidized by ROS (reactive oxygen species) in the course of atherosclerotic plaque formation. The macrophage cell line THP1, that is the precursor of atherosclerotic plaque, was cultured in a 48-well plate at the density of 2×10⁵ cells/well, for 24 hours. 160 nM of PMA was treated thereto to activate the cells. To form atherosclerotic plaque precursor cells that can cause arteriosclerosis, 10 μl/ml of PAF was treated to the cells 24 hours later, followed by transfection to induce over-expression of Grim19. After culturing the cells for 24 hours, oil red O staining was performed. At this time, the cells that turned to red after oil red O staining were the foam cells.

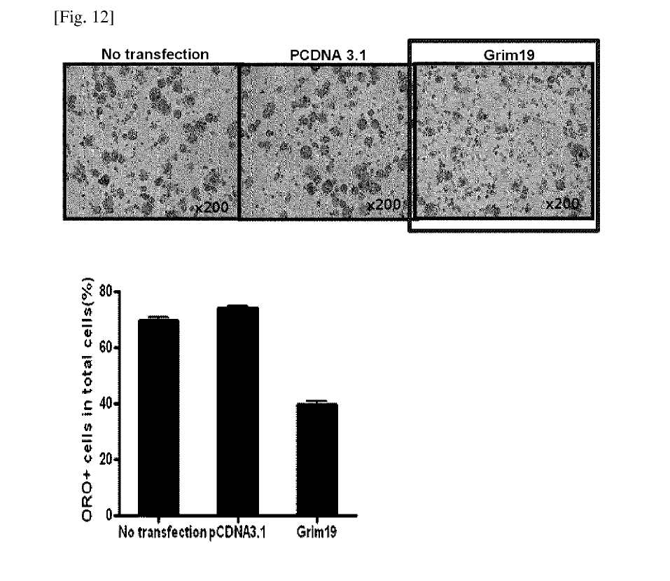

As a result, it was confirmed that the formation of foam cells was suppressed in the cells transfected with Grim19 (FIG. 12).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A pharmaceutical composition for the prevention or treatment of obesity or lipid-related metabolic disease comprising Grim19 protein as an active ingredient.

2. The pharmaceutical composition according to claim 1, wherein the lipid-related metabolic disease is selected from the group consisting of diabetes, hyperlipidemia, arteriosclerosis, hypertension, cardiovascular disease, fatty liver, obesity-mediated inflammatory disease, obesity-mediated autoimmune disease, and obesity-mediated cancer.

3. The pharmaceutical composition according to claim 1, wherein the Grim19 protein comprises the amino acid sequence represented by SEQ. ID. NO: 1.

4. The pharmaceutical composition according to claim 1, wherein the Grim19 has the effect of reducing body weight, fat cells, and total cholesterol, and converting white fat into brown fat.

5. A pharmaceutical composition for the prevention or treatment of obesity or lipid-related metabolic disease comprising the Grim19 protein of claim 1 or the polynucleotide encoding the same as an active ingredient.

6. The pharmaceutical composition according to claim 5, wherein the disease is selected from the group consisting of diabetes, hyperlipidemia, arteriosclerosis, hypertension, cardiovascular disease, fatty liver, obesity-mediated inflammatory disease, obesity-mediated autoimmune disease, and obesity-mediated cancer.

7. The pharmaceutical composition according to claim 5, wherein the polynucleotide comprises the nucleotide sequence represented by SEQ. ID. NO: 2 or NO: 3.

8. The pharmaceutical composition according to claim 5, wherein the polynucleotide is included in an expression vector.

9. The pharmaceutical composition according to claim 8, wherein the expression vector is a plasmid or a virus vector.

10. A method for screening a therapeutic agent for obesity or lipid-related metabolic disease comprising the following steps:
(a) culturing the Grim19 protein or the recombinant cells expressing the protein together with a candidate material; and
(b) measuring the effect of the candidate material on the activity or the intracellular level of Grim19 protein.

11. The method according to claim 10, wherein the activity or the intracellular level of Grim19 is measured by coimmunoprecipitation, RIA, ELISA, immunohistochemistry, Western blotting, or FACS.


SEQUENCE LISTING (35 sequences)

SEQ ID NO: 1 - DNA, 453 bp, Artificial Sequence
  A synthetic Grim19 cDNA sequence
ggatccagtatggcggcgtcgaaggtgaagcaggacatgcccccgccagggggctacggccccatcgactacaagcggaacctgccccgccggggactgtcggggtacagcatgtttgctgtgggcatcggggccttgatctttggctactggagaatgatgaggtggaaccaggagcgcaggcgcctgctgattgaggacttggaggccaggatcgccctcatgccgctcttccaggcagagaaggaccggaggaccctgcagattctccgggaaaacctggaggaggaagccatcatcatgaaggatgtgcccaactggaaggtgggcgagtctgtgttccataccacacgatgggtgccacccctcattggcgagatgtatgggttgcgcaccaaggaggagatgagcaatgccaacttcggcttcacctggtacacttagggcctcgag

SEQ ID NO: 2 - DNA, 453 bp, Artificial Sequence
  A synthetic codon optimized Grim19
ggatccagtatggctgccagcaaggtgaagcaggacatgccccctcctggcggctacggccccatcgactacaagagaaacctgcccagaagaggcctgagcggctacagcatgttcgccgtgggcatcggcgccctgatcttcggctactggagaatgatgagatggaaccaggagagacggagactgctgatcgaggacctggaggccagaatcgccctgatgcccctgttccaggccgagaaggacagaagaaccctgcagatcctgagagagaacctggaggaggaggccatcatcatgaaggacgtgcccaactggaaggtgggcgagagcgtgttccacaccacaagatgggtgcctcccctgatcggcgagatgtacggcctgagaaccaaggaggagatgagcaacgccaacttcggcttcacctggtacacctagggactcgag

SEQ ID NO: 3 - protein, 144 aa, Artificial Sequence
  A synthetic Grim19 peptide sequence
MAASKVKQDMPPPGGYGPIDYKRNLPRRGLSGYSMFAVGIGALIFGYWRMMRWNQERRRLLIEDLEARIALMPLFQAEKDRRTLQILRENLEEEAIIMKDVPNWKVGESVFHTTRWVPPLIGEMYGLRTKEEMSNANFGFTWYT

SEQ ID NO: 4 - DNA, 21 bp, Artificial Sequence
  A synthetic forward primer for C/EBP-a
caagaacagcaacgagtaccg

SEQ ID NO: 5 - DNA, 21 bp, Artificial Sequence
  A synthetic reverse primer for C/EBP-a
gtcactggtcaactccagcac

SEQ ID NO: 6 - DNA, 21 bp, Artificial Sequence
  A synthetic forward primer for Agt
gcaccctggtctctttctacc

SEQ ID NO: 7 - DNA, 21 bp, Artificial Sequence
  A synthetic reverse primer for Agt
tgtgtccatctagtcgggagg

SEQ ID NO: 8 - DNA, 19 bp, Artificial Sequence
  A synthetic forward primer for aP2
gatgcctttgtgggaacct

SEQ ID NO: 9 - DNA, 19 bp, Artificial Sequence
  A synthetic reverse primer for aP2
ctgtcgtctgcggtgattt

SEQ ID NO: 10 - DNA, 24 bp, Artificial Sequence
  A synthetic forward primer for PANK3
tgctctagtgtcccatttctgcct

SEQ ID NO: 11 - DNA, 21 bp, Artificial Sequence
  A synthetic reverse primer for RANK3
agctggaacagcaactaggaa

SEQ ID NO: 12 - DNA, 23 bp, Artificial Sequence
  A synthetic forward primer for Adiponectin
gtcagtggatctgacgacaccaa

SEQ ID NO: 13 - DNA, 19 bp, Artificial Sequence
  A synthetic reverse primer for Adiponectin
atgcctgccatccaacctg

SEQ ID NO: 14 - DNA, 23 bp, Artificial Sequence
  A synthetic forward primer for Resistin
aagaacctttcatttcccctcct

SEQ ID NO: 15 - DNA, 23 bp, Artificial Sequence
  A synthetic reverse primer for Resistin
gtccagcaatttaagccaatgtt

SEQ ID NO: 16 - DNA, 22 bp, Artificial Sequence
  A synthetic forward primer for LPL
ggaagagatttctcagacatcg

SEQ ID NO: 17 - DNA, 22 bp, Artificial Sequence
  A synthetic reverse primer for LPL
ctacaatgacattggagtcagg

SEQ ID NO: 18 - DNA, 22 bp, Artificial Sequence
  A synthetic forward primer for Leptin
cctcatcaagaccattgtcacc

SEQ ID NO: 19 - DNA, 22 bp, Artificial Sequence
  A synthetic reverse primer for Leptin
tctccaggtcattggctatctg

SEQ ID NO: 20 - DNA, 21 bp, Artificial Sequence
  A synthetic forward primer for UCP1
ctttgcctcactcaggattgg

SEQ ID NO: 21 - DNA, 21 bp, Artificial Sequence
  A synthetic reverse primer for UCP1
actgccacacctccagtcatt

SEQ ID NO: 22 - DNA, 20 bp, Artificial Sequence
  A synthetic forward primer for Elvol3
cgggttaaaaatggacctga

SEQ ID NO: 23 - DNA, 20 bp, Artificial Sequence
  A synthetic reverse primer for Elvol3
ccaacaacgatgagcaacag

SEQ ID NO: 24 - DNA, 20 bp, Artificial Sequence
  A synthetic forward primer for PGC1-alpha
gtcaacagcaaaagccacaa

SEQ ID NO: 25 - DNA, 20 bp, Artificial Sequence
  A synthetic reverse primer for PGC1-alpha
tctggggtcagaggaagaga

SEQ ID NO: 26 - DNA, 23 bp, Artificial Sequence
  A synthetic forward primer for FGF21
cctctaggtttctttgccaacag

SEQ ID NO: 27 - DNA, 19 bp, Artificial Sequence
  A synthetic reverse primer for FGF21
aagctgcaggcctcaggat

SEQ ID NO: 28 - DNA, 21 bp, Artificial Sequence
  A synthetic forward primer for Cidea
gccgtgttaaggattctgctg

SEQ ID NO: 29 - DNA, 21 bp, Artificial Sequence
  A synthetic reverse primer for Cidea
tgctcttctgtatcgcccagt

SEQ ID NO: 30 - DNA, 20 bp, Artificial Sequence
  A synthetic forward primer for Cox7a1
agaaaaccgtgtggcagaga

SEQ ID NO: 31 - DNA, 20 bp, Artificial Sequence
  A synthetic reverse primer for Cox7a1
cagcgtcatggtcagtctgt

SEQ ID NO: 32 - DNA, 20 bp, Artificial Sequence
  A synthetic forward primer for PRDM16
gacattccaatcccaccaga

SEQ ID NO: 33 - DNA, 20 bp, Artificial Sequence
  A synthetic reverse primer for PRDM16
cacctctgtatccgtcagca

SEQ ID NO: 34 - DNA, 20 bp, Artificial Sequence
  A synthetic forward primer for Cytochrom C1
gctacccatggtctcatggt

SEQ ID NO: 35 - DNA, 20 bp, Artificial Sequence
  A synthetic reverse primer for Cytochrom C1
catcatcattagggccatcc